(19) United States
(12) Patent Application Publication
(10) Pub. No.: US 2026/0102278 A1
(43) Pub. Date: Apr. 16, 2026

(54) COOLING THERAPY APPARATUS

(71) Applicant: Nice Recovery Systems LLC, Boulder, CO (US)

(72) Inventors: Michael Ross, Boulder, CO (US); Joel Meisinger, Boulder, CO (US); Gerry Taylor, Boulder, CO (US)

(21) Appl. No.: 19/407,286

(22) Filed: Dec. 3, 2025

Related U.S. Application Data
(63) Continuation-in-part of application No. 29/957,328, filed on Aug. 13, 2024, now Pat. No. D1110526.
Application No. 29/957,328 is a continuation of application No. 18/221,237, filed on Jul. 12, 2023.

(51) Int. Cl. A61F 7/00; A63B 22/06

(52) U.S. Cl. CPC A61F 7/007; A63B 22/0664; A61F 2007/0036; A61F 2007/0075; A61F 2007/0087; A63B 2225/66

(57) ABSTRACT

An apparatus is described that provides cooling therapy for a user of the apparatus. A heat exchanger and/or thermoelectric module in the device, such as a Peltier cooling module, promotes cooling of one or more thermally conductive portions of the device and objects (such as the user's hands holding the device) in close proximity to the thermally conductive portions. A user may place one's palm(s) on the thermally conductive portion(s) to cool the user's palms. As a user's palms are cooled over a time period of use of the device of the present invention, the user's body temperature is also cooled, which reduces negative impacts of body heat on the user's muscles, and thereby enhances physical performance by the user for an added period of time.

### CROSS-REFERENCE TO RELATED APPLICATIONS

This application is a continuation in part application claiming priority to U.S. application Ser. No. 29/957,328 , filed Aug. 13, 2024, which is a continuation of U.S. application Ser. No. 18/221,237 , filed on Jul. 12, 2023. The disclosure of each aforementioned application is incorporated by reference as if fully recited herein.

### TECHNICAL FIELD

The present invention relates generally to a device for cooling therapy, and more particularly to a hand-held device for cooling the palms of a user's hands to thereby improve performance of a physical endeavor, such as physical exercise. In one example embodiment, a thermally conductive device is configured with a plurality of air inlets and outlets adapted to permit air to be circulated within the device. One or more thermally conductive portions of the device may be cooled by a thermoelectric module for cooling objects in close proximity thereto. In the aforementioned example embodiment, a user may place one or both of the user's palms on the device to cool the body temperature of the user.

### BACKGROUND AND SUMMARY OF THE INVENTION

Muscle strength, endurance, performance and the like may be adversely impacted by the accumulation of internal heat in body tissues during and/or after exercise. By way of example and not limitation, it is known that performance of exercises may result in significant accumulation of internal heat in body tissues, temporarily reducing muscle performance and endurance. Although excess internal heat in body tissues may decrease after an extended amount of time has passed following the completion of exercise(s), it may be preferable to remove excess internal heat from the body sooner. By way of example and not limitation, in a situation where one desires to engage in continued and/or subsequent exercises, and significant internal heat in body tissues has accumulated, muscle strength, endurance, performance and the like may be improved to promote said continued and/or subsequent exercises by cooling the body.

It is known that heat may be extracted from the body by transferring heat away from certain skin surfaces substantially free of hair. As a specific example, on the surface of one's hands (as well as the bottom of one's feet and one's upper face), glabrous skin is present, and temperature regulation of glabrous skin is believed to affect core body temperature. A known device for extracting body heat in an attempt to, e.g., improve muscle strength, endurance, performance and the like, includes a cooling mitten having cool water pathways adapted to circulate cool water to substantially hairless skin of the palms, soles and/or face to cool blood at surfaces thereof. The aforementioned skin may comprise relatively high volumes of blood, and the known device may include a vacuum for increasing blood volume at said surfaces. Disadvantages of the known device include by way of example and not limitation, that it is highly complex, requires a large amount of storage and operation space, is expensive to end users, and the effects of using a vacuum may not be predictable for different users. The necessity for fluid pathways and pressure regulation requires the known device to provide substantial space and material to permit said fluid pathways and pressure regulation. Another known device for extracting body heat in an attempt to, e.g., improve muscle strength, endurance, performance and the like, includes a metallic bar filled with a fluid such as water and preferably refrigerated to provide a cool surface to contact skin. Disadvantages of the known device include that temperature of the device rapidly increases as fluid therein is warmed by body heat, and the device requires refrigeration.

The aforementioned shortcomings speak to the need for a small, lightweight, cost-effective device wherein cool temperature of a surface of the device is maintained over time to promote heat transfer from a body. In view of this, it is beneficial to have a cooling therapy device and method involving a thermally conductive surface. Temperature of the surface may be regulated by one or more airflow pathways. Alternatively, or additionally, temperature of the surface may be regulated by a heat exchanger, thermoelectric module, some combination thereof, or the like. An exemplary embodiment of the present invention is adapted to extract body heat from a palm of a user without requirements for liquid and/or vacuums.

According to the present invention in one aspect, an exemplary cooling therapy device comprises a rigid, thermally conductive surface. The rigid, thermally conductive surface may comprise lightweight, relatively inexpensive material. By way of example and not limitation, a portion of the thermally conductive surface may comprise aluminum, and/or any number of other lightweight, relatively inexpensive metals, including but not limited to combinations thereof. It will be apparent to one of ordinary skill in the art that any number of different rigid, thermally conductive materials, including but not limited to combinations thereof, may be employed without departing from the scope of the present invention.

An exemplary device of the present invention may be sized appropriately for contact with one or both palms and fingers of a user. It will be apparent to one of ordinary skill in the art that exemplary embodiments may be formed in different shapes and sizes suitable for contact with a user's palm(s). It will also be apparent to one of ordinary skill in the art that finger contact is not required to permit body heat transfer from a user.

The exemplary device may include a battery pack power supply positioned between structural brackets inside of the rigid, thermally conductive outer enclosure surface. The battery pack may comprise one or more rechargeable batteries regulated by an exemplary battery management system (“BMS”). An assembly for securing the battery pack and BMS may include through bolts, fasteners, fastener channels, and the like. The battery pack may be adapted to satisfy power requirements for one or more fans, heat exchangers, control units, thermoelectric modules, some combination thereof, or the like. It will be apparent to one of ordinary skill in the art that there are different methods and/or materials available for regulating temperature of the thermally conductive surface without departing from the scope of the present invention.

According to the present invention in another aspect, an exemplary cooling therapy device comprises a thermally conductive outer enclosure having a plurality of ambient air inlets and cooled air outlets adapted to permit one or more fans to circulate air within the device for cooling thereof. A user may place one or both of the user's palms on a portion of the outer enclosure of the device to contact the cold surface and/or encounter the cool air to thereby modify the body temperature of the user. Various other exemplary embodiments may be positioned on/within a handle, such as the handle of an aerobic or anaerobic exercise device.

According to the present invention in yet another aspect, an exemplary hand-held cooling therapy device comprises a solid, thermally conductive outer enclosure having at least one ambient air inlet and at least one air outlet for discharging waste heat. The device may include an interior portion comprising a power supply secured within the outer enclosure, and at least one temperature modification module secured within the outer enclosure. The at least one temperature modification module may be configured to generate a cooler temperature at a first portion (e.g., a first hemispherical aluminum end piece) of the outer enclosure than a temperature of the ambient air. The device may be configured to be hand-held such that at least one of the palms of a user's hands may contact the first portion to cool the at least one palm. The device may also include a second temperature modification module configured to generate a cooler temperature at a second portion (e.g., a second hemispherical aluminum end piece) of the outer enclosure compared to the temperature of the ambient air. The first and second portions may be located opposite one another.

The device may also include control circuitry configured to cause the first (and/or second) portion to reach a first temperature after a first amount of time, and to reach a second temperature after a second amount of time following the first amount of time. The second temperature may be different (e.g., warmer) than the first temperature and cooler than the temperature of the ambient air. The second temperature may be cooler than 25° C. The temperature modification module may be a Peltier or other thermoelectric module. The thermoelectric module may be configured to produce a plurality of discrete cooling cycles, each cooling cycle occurring for a finite period of time. The control circuitry may be configured to cause the power supply to initially provide at least about 50 W to the temperature modification module during one of the cooling cycles (e.g., to achieve a first temperature), and at a subsequent time in the one of the cooling cycles, provide about 20-30 W to the temperature modification module (e.g., to achieve a second temperature). The device may also include a temperature sensor for monitoring a body temperature of a user. The control circuitry may be configured to cause the temperature modification module to be adjusted when a body temperature measured by the temperature sensor falls above or below a threshold.

It will be apparent to one of ordinary skill in the art that exemplary embodiments of the present invention provide a number of different advantages. Exemplary embodiments decrease internal heat accumulated in body tissues such as to, by way of example and not limitation, promote muscle strength, endurance, performance, and the like. An exemplary embodiment of the present invention is useful for promoting longer periods of high intensity exercise for a user, which may result in part from a user experiencing less bodily inflammation due to lower body temperature than would occur without the use of the invention.

The present invention provides a number of improvements over known devices for extracting/lowering body heat. By way of example and not limitation, the present invention is preferably small, lightweight, and cost effective, making the therapy device accessible to more users. The present invention is simple to use and simple to maintain. Operating the present invention is as simple as turning the device on and holding it in one's hand(s). Maintaining the invention may involve recharging and/or replacing one or more batteries thereof as said batteries drop below a minimum threshold state of charge.

### BRIEF DESCRIPTION OF THE DRAWINGS

Novel features and advantages of the present invention, in addition to those expressly mentioned herein, will become apparent to those skilled in the art from a reading of the following detailed description in conjunction with the accompanying drawings. The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 illustrates a top plan view of an exemplary cooling therapy device of the present invention;

FIG. 2 illustrates a perspective view of the exemplary cooling therapy device of FIG. 1;

FIG. 3 illustrates an exploded perspective view of a portion of the exemplary cooling therapy device of FIG. 1;

FIG. 4 illustrates a cross-section view of an exemplary power module of the exemplary cooling therapy device of FIG. 1;

FIG. 5 illustrates another perspective view of the exemplary cooling therapy device of FIG. 1;

FIG. 6 illustrates an exercise apparatus grip handle adapted to receive another exemplary palm cooling device of the present invention;

FIG. 7 illustrates another top plan view of the exemplary cooling therapy device of FIG. 1;

FIG. 8 illustrates an exemplary diagrammatic view of a high-level schematic electronics layout for the present invention;

FIG. 9 illustrates a front perspective view of another exemplary cooling therapy device of the present invention, wherein both of a user's palms are positioned at the device;

FIG. 10 illustrates another perspective view of the device of FIG. 9, wherein one palm of the user is positioned at the device;

FIG. 11 illustrates a table indicating exemplary temperature modification module power input over time for an exemplary cooling therapy device of the present invention;

FIG. 12 illustrates exemplary circuitry for powering on and off the device of FIG. 9;

FIG. 13 illustrates exemplary logic for user interaction with an exemplary cooling therapy device of the present invention;

FIG. 14 illustrates exemplary circuitry of a Peltier module for cooling a conductive face of the device of FIG. 9;

FIG. 15 illustrates a front view of an exemplary PCBA for implementing at least a portion of the circuitry of FIG. 14;

FIG. 16 illustrates a front view of an exemplary PCB for implementing at least a portion of the circuitry of FIG. 14;

FIG. 17 illustrates another front view of the PCBA of FIG. 15;

FIG. 18 illustrates a rear view of the PCB of FIG. 16; and

FIG. 19 illustrates exemplary control circuitry of the device of FIG. 9.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

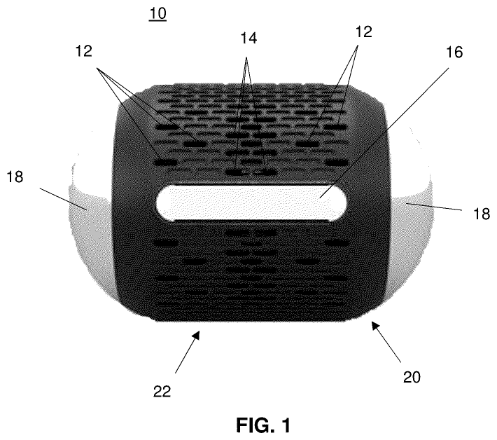

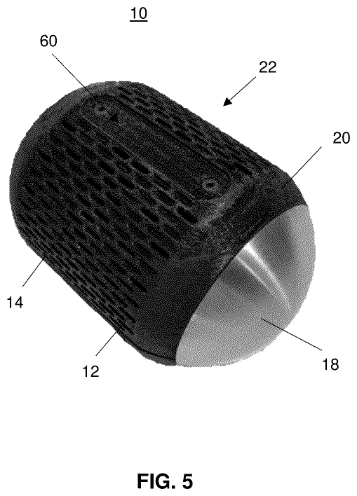

Referring now to FIGS. 1-5 and 7, an exemplary cooling therapy device 10 is shown. This particular embodiment includes a thermally conductive outer enclosure 20 configured with a plurality of ambient air inlets 12 and cool air outlets 14 adapted to permit one or more fans 26 to circulate air within the device 10 for cooling thereof. In this particular embodiment, a user may place one or both of the user's palms on a portion 22 of the outer enclosure 20 to modify the body temperature of the user. Specifically, as high volumes of blood flow through the user's palms positioned on the device 10, the user's body temperature may be reduced. The reduction of the user's body temperature may promote, for example, muscle strength, endurance, performance, and the like, such as for continued high intensity exercise for an additional period of time as desired by the user. Without body temperature cooling, accumulation of internal heat in user muscle tissue may adversely affect muscle strength, endurance, performance, and the like, due to such effects as inflammation in the body or muscle fatigue. The outer enclosure 20 may preferably comprise lightweight, relatively inexpensive, rigid material. The thickness of the outer enclosure 20 may be consistent with optimizing volumetric heat capacity.

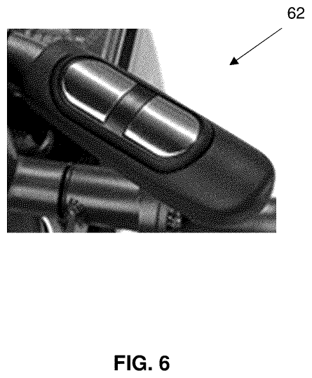

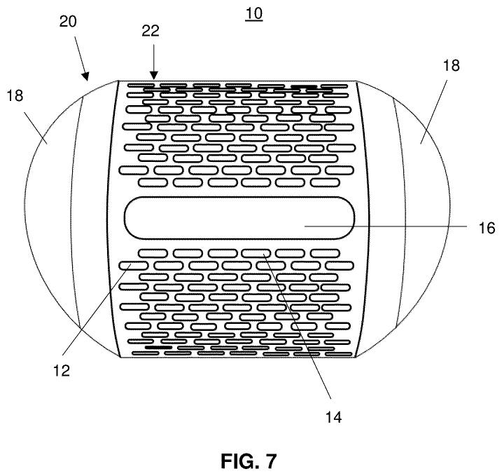

Referring to FIGS. 6-7, the exemplary cooling device 10 may be positioned on or made an integral part of a handle 62, such as the handle of an aerobic or anaerobic exercise device. It will be apparent to one of ordinary skill in the art that an exemplary cooling therapy device may be positioned on different exercise devices, such as aerobic (e.g., stationary bikes, elliptical machines, rowing machines, treadmills, and the like) and/or anaerobic exercise equipment (e.g., Olympic bars, curl bars, dumbbells, and the like). An exemplary cooling therapy device of the present invention may specifically be positioned on (or formed as a part of) a handle, grip, monitor, some combination thereof, or the like of said exercise equipment/apparatus in a location where the user may readily grip the portion having the invention thereon or therein, to promote heat transfer from a user's palm during an exercise (e.g., to promote improved performance). In this embodiment, the invention may be comprised of the same components as in the stand-alone embodiment, except it would be configured to fit in or fit on a portion of an exercise apparatus to be used by a user while exercising on or with the exercise apparatus.

Referring again to FIGS. 1-5 and 7, a user may position the palm(s) of one or both hands on a substantially thermally conductive mid-portion 22 of the exemplary cooling device 10. Faces 18 may be positioned on each side of the mid-portion 22, and the faces 18 and mid-portion 22 may collectively define an outer enclosure 20 of the device 10. The faces 18 may also be thermally conductive (e.g., comprise aluminum casings). The outer enclosure 20 may also include a label receptacle 60 adapted to receive a label plate or brand marking (e.g., 16), although such is not required. The label receptacle 60 may be configured to receive and secure a label plate 16 by way of one or more fasteners being secured through one or more corresponding apertures, or by way of an adhesive for example. A plurality of airflow outlets 14 may be centrally located across the surface of the mid-portion 22. A plurality of airflow inlets 12 may be located apart from the outlets 14 on each side of the mid-portion 22. It will be apparent to one of ordinary skill in the art that the specific shape, location and number of the various airflow inlets 12 and outlets 14 may be varied without departing from the scope of the present invention. It will also be apparent to one of ordinary skill in the art that an exemplary device may be defined by different shapes and/or sizes without departing from the scope of the present invention.

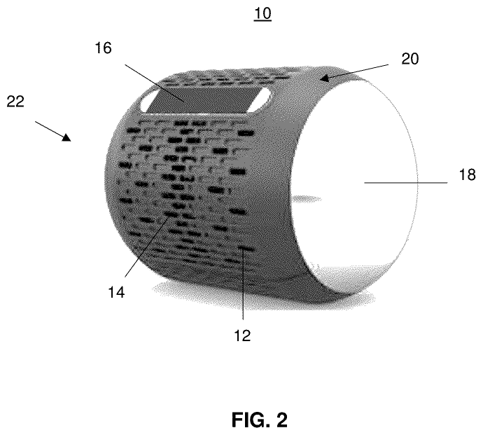

Referring specifically to FIGS. 2-4, a control module receptacle 32 may permit various objects within an interior 23 of the device 10 to be secured therein. A pin heat exchanger 36 and control circuitry 38 of the device 10 may be positioned within or proximate to the control module receptacle 32 (e.g., by way of fasteners 34). In the embodiment shown, the pin heat exchanger 36 and control circuitry 38 is secured between the control module receptacle 32 and a side attachment apparatus 40. The side attachment apparatus 40 shown is configured to immobilize one or more interior components (e.g., 36, 38) proximate to an aluminum casing face 18. Here, an interior frame of the side attachment apparatus 40 is configured to receive and secure the control circuitry 38. Operation of the various electronic components of the device, including a fan 26, may be regulated by the control circuitry 38. The control circuitry 38 may specifically dictate fan 26 speed, duration of fan 26 activity, heat exchanger 36 activity, thermoelectric module activity (if applicable), some combination thereof, or the like. A user may interact with the control circuitry 38 by way of a remote, control board on the device 10 exterior 20, some combination thereof, or the like. As a specific, non-limiting example, a user may select from within a predetermined temperature range (sufficient for safely extracting body heat) a specific operating temperature at the outer enclosure 20.

A user may also engage electronic controls of the device to dictate on and off status of the device. An exemplary device may be configured to operate no longer than a maximum amount of time during a single cooling therapy session in order to reduce or prevent the lowering of core body temperatures too low. Certain exemplary devices may include an electronic interface for communicating temperature (e.g., measured by a temperature sensor positioned in the device interior), state of charge, other diagnostic information, some combination thereof, or the like to a user. The temperature sensor may monitor temperature of the device when the device is in use, and operation of the device may be controlled at least in part based on the temperature readings of the temperature sensor.

The aforementioned interface may be engaged by a user to control certain therapy session parameters. Certain exemplary devices may be configured with an internal processor, or may be in electronic communication with an external processor to promote temperature regulation and the communication of device information. It will be apparent to one of ordinary skill in the art that there are a number of different methods/techniques available for regulating interior components of an exemplary device without necessarily departing from the scope of the present invention.

An attachment apparatus 28 may permit securement of the fan 26 proximate to the control module receptacle 32 within an interior 23 of the device 10. Specifically, the fan 26 may be secured by way of one or more fasteners 24 being positioned in apertures of the attachment apparatus 28. The control module receptacle 32 and side attachment apparatus 40 may each be secured to inner walls (not shown) of the device 10 outer enclosure 20, such as by clips, fasteners, some combination thereof, or the like. It will be apparent to one of ordinary skill in the art that exemplary components may be secured within an exemplary device by way of different connection methods or techniques without departing from the scope of the present invention.

In the embodiment shown, movement of the fan 26 causes ambient air to be drawn into the device 10 interior through air inlets 12. Said air may be directed from the inlets 12 to the heat exchanger 36 by way of a number of inbound air flow channels. Here, the heat exchanger 36 is configured to decrease the temperature of said air. Conductive surfaces 18 of the heat exchanger 36 may be cooled by a number of different thermoelectric modules, convection-based cooling techniques, cross-fluid heat exchange, some combination thereof, or the like. A thermoelectric module may regulate temperature(s) within the heat exchanger 36, and may be controlled by the control circuitry 38. A thermoelectric module may include a Peltier module. The various electronic components of the device 10 may be powered by a power module 30. The power module 30 may comprise a battery, power board, some combination thereof, or the like. It will also be apparent to one of ordinary skill in the art that different air temperature modification techniques involving a heat exchanger, heat sink, thermoelectric module, fluid crossflow heat transfer device, some combination thereof, or the like (individually or collectively, “air temperature modification devices”) may be employed for decreasing air temperature without departing from the scope of the present invention.

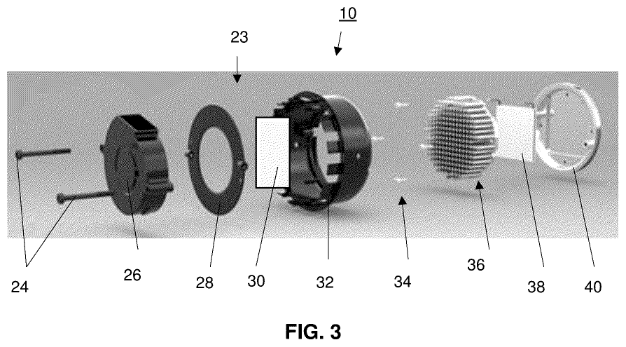

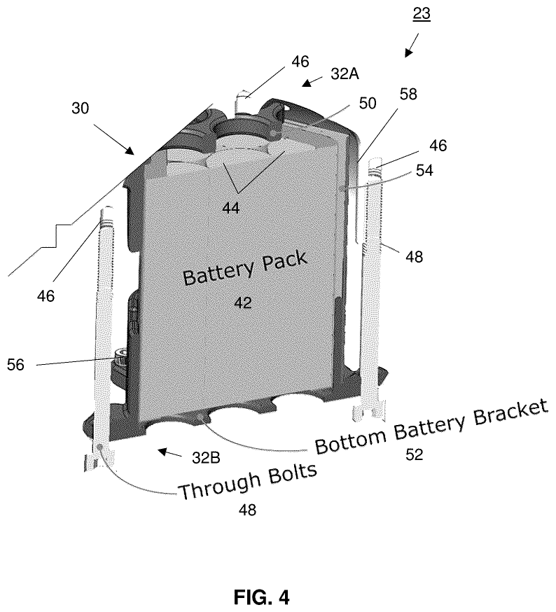

Referring now specifically to FIGS. 3-4, an exemplary power module 30 positioned in an interior 23 of the device 10 comprises a rechargeable battery pack 42. Electronic communication between the battery pack 42 and each of various electronic components (e.g., 26, 36, 38) of the device 10 may be established by a wire. A charging port of the battery pack 42 may permit a user to connect a charging device to the battery pack 42 to recharge one or more batteries 44 of the battery pack 42. It will be apparent to one of ordinary skill in the art, however, that rechargeable batteries are not necessarily required in certain other embodiments. Electronic component (e.g., 26, 36, 38) power requirements may be satisfied by a number of different power sources without departing from the scope of the present invention. Batteries, whether rechargeable or single use, are not necessarily required to satisfy power requirements for the present invention. Various battery technologies from alkaline to lithium ion and many more varieties may be suitable for the present invention.

In this particular embodiment, the battery pack 42 is secured between a top battery bracket 50 and a bottom battery bracket 52 within the control module receptacle 32 of the interior 23. It will be apparent to one of ordinary skill in the art that the particular sizes and shapes of battery brackets shown are merely illustrative, and a number of different materials and/or mechanisms may be employed to secure an exemplary battery pack within the interior without departing from the scope of the present invention. The battery pack 42 may comprise a number of batteries 44. The battery pack 42 may be regulated by an exemplary BMS 54. The BMS 54 may be configured to ensure the batteries 44 operate within a safe operating area, monitor voltage, monitor battery temperature, monitor current, monitor state of charge, some combination thereof, or the like. A portion 58 of the power module 30 may be configured to receive and secure the BMS 54.

The top 50 and bottom 52 battery brackets may each be sized to maintain and restrict movement of the battery pack 42 within the interior 23 of the device 10. The bottom battery bracket 52 may be secured (e.g., by way of one or more fasteners 56) to a lower portion 32B of the control module receptacle 32. Specifically, the one or more fasteners 56 may be positioned through an aperture of the bottom battery bracket 52 and into a respective fastener channel of the lower portion 32B of the control module receptacle 32. The bottom battery bracket 52 may further be secured within the interior 23 of the device 10 by positioning each of one or more through bolts 48 through an aperture of the bottom battery bracket 52 and into a respective fastener channel 46 at an upper portion 32A of the control module receptacle 32. A user may be permitted to loosen each of the one or more through bolts 48 to disengage said bolts 48 from the fastener channels 46 (e.g., to replace a battery pack 42).

The top battery bracket 50 may be secured to the upper portion 32A of the control module receptacle 32 by positioning each of one or more pegs, fasteners, or the like in each of a top battery bracket aperture (not shown) and an upper portion 32A channel (not shown). It will be apparent to one of ordinary skill in the art that the specific configurations for securing interior components of the device illustrated and described herein are in no way intended to be exhaustive of the scope of the present invention. Any number of different materials and/or mechanisms for securing electronic components within a device interior may be employed without departing form the scope of the present invention.

Referring specifically to FIGS. 2-3, as said air flows across said conductive surfaces of the heat exchanger 36, the temperature of said air may be significantly reduced. The reduced-temperature air may then be directed to air outlets 14 by way of a number of outbound air flow channels. The reduced temperature air may then be discharged through said outlets 14, and may thereafter contact a user's palms to reduce body temperature of the user. The reduced temperature air may also cool a conductive surface of the device 10 exterior 20 to further contribute to reducing body temperature of the user (e.g., by way of heat removal from the user's palm(s)). As a non-limiting example, a user may place a first hand on a first side of the device 10 exterior 20, and a second hand on a second side of the device 10 exterior 20 such that both palms of the user are directly exposed to cold air being discharged from outlets 14, and a cool surface of the mid-portion 22. It will be apparent to one of ordinary skill in the art that the specific arrangement of interior and exterior components shown is merely illustrative, and variations may be made without necessarily departing from the scope of the present invention.

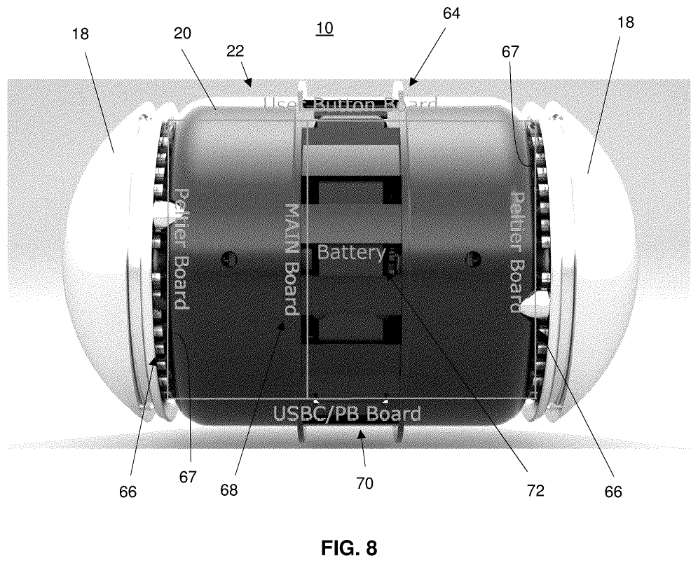

Referring now to FIG. 8, an example high-level electrical schematic diagram is shown overlayed on an exemplary embodiment of the cooling therapy device 10 of the present invention for cooling thermally conductive faces 18. An electronics board 64 for operation button controls is indicated, as is a main processor control board 68, a Peltier control board 67 (for controlling Peltier module 66), and a USBC/PB Board 70, powered by the enclosed battery or battery pack 72 within the device 10. The invention electronics may be controlled by a microcontroller which may be in electronic communication with: an LED driver circuit which may be incorporated for operational signals; a battery charging circuit and battery connector; a power push button controller circuit; a power circuit; one or more Peltier power circuit(s), a Peltier engine feedback circuit, and a Peltier current feedback circuit. One or more of the aforementioned electronic components may be positioned inside a mid-portion 22 of the outer enclosure 20.

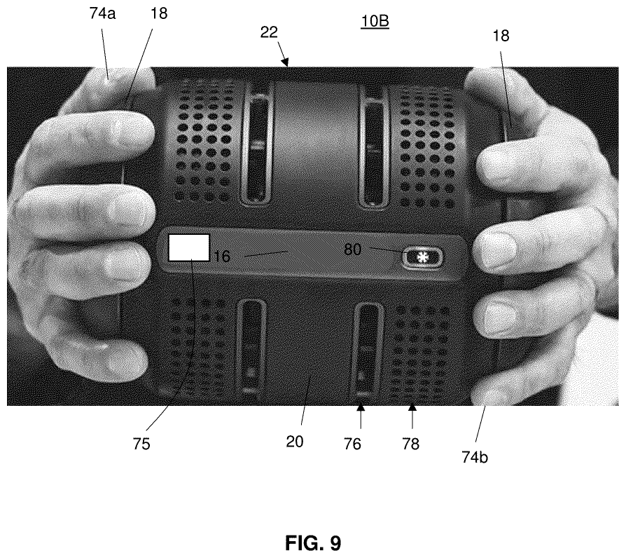

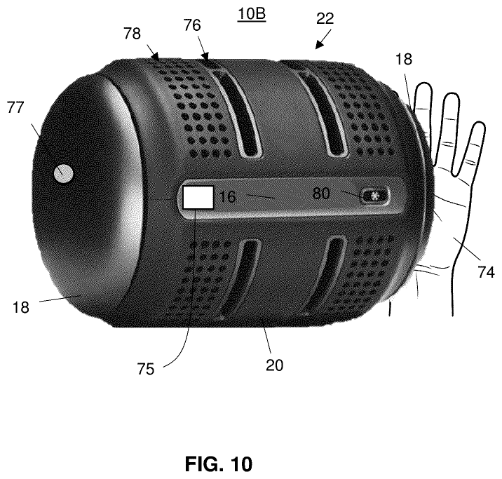

Referring now to FIGS. 9-10, another exemplary cooling therapy device 10B is shown. A user may place one hand (e.g., 74 as shown in FIG. 10) or both hands (e.g., 74a-b as shown in FIG. 9) on a solid, thermally conductive outer enclosure 20 of the device 10B. Thermally conductive end faces 18 of the device 10B may be provided for the user to place one or both hands. In this particular embodiment, there is a left-hand and right-hand side thermally conductive face 18, each of which is hemispherical in shape and comprises solid, thermally conductive material (e.g., aluminum, another metal, another thermally conductive material, or some combination thereof). The number, shape, location, dimensions, and/or material composition of one or more thermally conductive portions of an exemplary device may be varied. Likewise, the shape, dimensions, and/or material composition of the outer enclosure 20 may be varied.

Each thermally conductive face 18 of the device 10B may be configured to be cooled to cool glabrous skin of a user (e.g., skin at the palm of the user's hand 74). The thermally conductive face(s) 18 may be located proximate to a temperature modification module (a Peltier module, another thermoelectric module, a heat exchanger, some combination thereof, or the like) (for example, the Peltier module 66 shown in FIG. 8 may be employed to cool an end face 18 of FIGS. 9-10). The temperature modification module(s) may be secured within the outer enclosure 20, and may be regulated by control circuitry of the device 10B. Variations to the type, number and/or location of the temperature modification modules may be made without departing from the scope of the present invention.

The outer enclosure 20 may include at least one ambient air inlet and at least one air outlet. The at least one ambient air inlet may allow for ambient air to be drawn into the device 10B to remove heat from the temperature modification module(s) (e.g., by blowing air at fins on the hot side of a Peltier module such as those shown at 66 in FIG. 8, fins of another thermoelectric module, a portion of a heat exchanger, some combination thereof, or the like) and/or other interior components. Airflow in the device 10B may transfer heat away from the temperature modification module(s) and/or other interior components. The at least one air outlet may allow waste heat to be discharged from the device 10B. The at least one ambient air inlet may be located at a first end of an airflow pathway, and the at least one ambient air outlet may be located at a second end of the airflow pathway. The airflow pathway may be defined by one or more interior structures of the device 10B, and the airflow may be driven by one or more fans (e.g., located in the device 10B interior).

In the embodiment shown, a first number of openings 76 and a second number of openings 78 are shown at a mid-portion 22 of the outer enclosure. The first number of openings 76 may define a plurality of air outlets, and the second number of openings 78 may define a plurality of ambient air inlets, or vice versa. The shape, number, location, dimensions, and/or arrangement of ambient air inlets and air outlets may be varied. The temperature modification module(s), fan(s), and/or other device 10B electronic components may be powered by a power supply (e.g., one or more batteries) secured within the outer enclosure 20. The one or more temperature modification modules may be configured to generate a cooler temperature at one or both thermally conductive faces 18 of the outer enclosure 20 than a temperature of the ambient air.

To achieve said cooler temperature at one or both thermally conductive faces 18, the user may press a power button 80 of the device 10B. The power button 80 may be located at a label plate 16, although such is not required. The user may press the power button 80 a second time to cut power to the one or more temperature modification modules, which may cause the temperature of the thermally conductive faces 18 to increase towards the ambient air temperature. Alternatively or additionally, a digital display screen (e.g., at controller 75) may be provided to allow a user to interact with a user interface visible at the digital display screen to power on and off the device 10B, and/or to regulate temperature of the device 10B.

Referring to FIGS. 9-11, after the device 10B is powered on (e.g., by the power button 80) at an initial time tₒ, the power supplied from the power supply to a temperature modification module of the device 10B may increase from 0 W to about 50-60 W when a first amount of time passes Δt₁ from the initial time tₒ. After Δt₁, the temperature of at least one thermally conductive face 18 of the device 10B may decrease ΔT₁ from an initial temperature Tₒ to a stage 1 temperature Tₛ₁. Tₛ₁ may be sufficient to cause the user's body temperature to decrease. When a second amount of time passes Δt₂ from when approximately 50-60 W is supplied to the temperature modification module, the power supplied to the temperature modification module may be decreased to between about 20-30 W (e.g., approximately 20-25 W). After Δt₂, the temperature of the at least one thermally conductive face 18 may change (e.g., increase) ΔT₂ from Tₛ₁ to a stage 2 temperature Tₛ₂ (e.g., Tₛ₁>Tₛ₂). Alternatively, the temperature of the conductive faces 18 may be constant after Tₛ₁ is reached (i.e., Tₛ₁=Tₛ₂). The initial power input of approximately 50-60 W may permit the conductive faces to reach Tₛ₁ and/or Tₛ₂, and the drop in power input to about 20-30 W (e.g., about 20-25 W) may prevent the conductive faces from dropping below a threshold temperature (e.g., below 50° F.).

Tₛ₁ and/or Tₛ₂ may be low enough to temporarily maintain a lower user body temperature, but high enough to eliminate safety risks to the user (e.g., the risks of reaching lower than desired body temperature, harming glabrous skin, some combination thereof, or the like). Following Δt₂, the power supplied to the temperature modification module may be maintained at approximately 20-30 W for an amount of time Δt₃ (e.g., to maintain the temperature of the thermally conductive face 18 at Tₛ₂ for the amount of time Δt₃). Following Δt₃, the user may power off the device, or the device may automatically power down at a final time t_{f}. At t_{f}, the power supplied to the temperature modification module may be 0 W, and the temperature of the thermally conductive face 18 may increase ΔT₃ from Tₛ₂ towards the initial temperature Tₒ of the face 18.

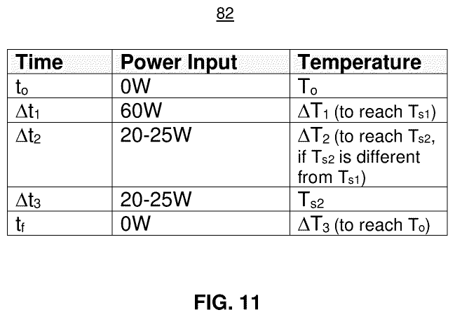

Exemplary power input and temperature variations over time are illustrated by table 82 in FIG. 11. Although not required, Tₒ may be room temperature (approximately 70-75°F.). The initial significant temperature differential provided by Tₛ₁ at the interface between the face 18 and a user's glabrous skin may be required to lower the user's body temperature. When Tₒ is approximately 70-75° F. (e.g., 72° F.), it may take about 90-120 seconds for the conductive faces 18 to reach Tₛ₁ and/or Tₛ₂. For example, Δt₁ may be about 90 seconds, Δt₂ may be about 30 seconds or less, and Δt₃ may be a user specified amount of time. It may take longer for the conductive faces 18 to reach Tₛ₁ and/or Tₛ₂ when Tₒ is above room temperature. Tₛ₂ may be between about 35° F. and about 60° F. (e.g., between about 50-60° F.). This range may be advantageous for maximizing cooling benefits to a user without causing the user's body temperature to drop below a desired level. Tₛ₁ may be different from or equal to Tₛ₂. Having the thermally conductive faces 18 set to a temperature of 57° F. may provide several advantages. For example, such may promote battery life, and may prevent a thermal bounce from a user picking up a device having conductive faces 18 initially at 57° F. from raising face 18 temperature above 60° F. (i.e., there may be about a 3° F. buffer, since thermal bounce is often about 1-2° F. at a conductive surface). Cooling benefits may be reduced when face 18 temperature rises above 60° F. The length of a therapy session may be about 90-120 seconds. The length of a therapy session may be longer than 120 seconds. The device 10B is not limited to any particular range of therapy session lengths.

The device 10B may include an external user controller 75, which may include one or more buttons, screens, dials, knobs, digital displays, some combination thereof, or the like. The device 10B is not limited to any particular user controller type, size and/or location. A user may engage the power button 80 and/or controller 75 (e.g., the controller 75 may include a button, and the user may press the button) to start a timer (not shown in FIG. 9-11). The user may also engage the controller 75 to dictate the specific temperature of the conductive faces 18. As a specific, non-limiting example, the user may specify that after 90 seconds from start, the conductive faces 18 are to drop to a Tₛ₁ around 50° F., and then after 120 seconds from start, the conductive faces are to be maintained at a Tₛ₂ around 57° F. As an alternative non-limiting example, the user may specify that after 90 seconds from start, the conductive faces are to drop to and be maintained at 57° F.

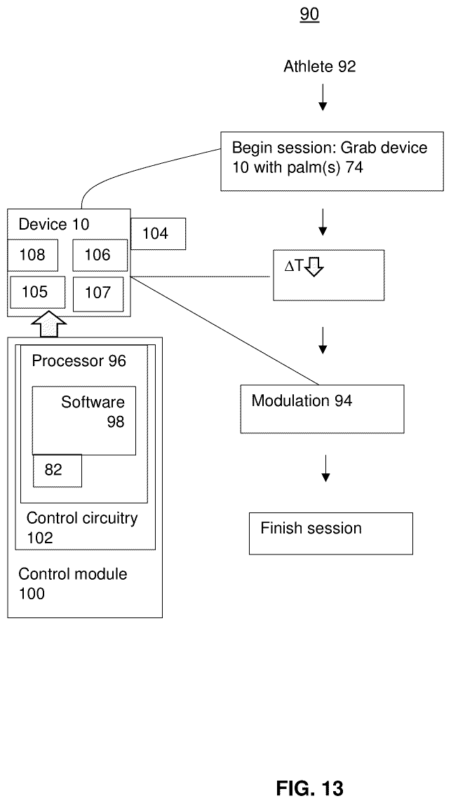

Referring to FIG. 13, the timer 107 may be positioned inside the device 10, and may be in electronic communication with control circuitry 102 of the device 10. The control circuitry 102 may also be located within an outer enclosure of the device 10, and may be in electronic communication with a user interface 108. The timer 107 may cause the unit 10 to vibrate each time a certain amount of time (e.g., approximately 30 seconds) passes while the device 10 is active. In doing so, the timer 107 may allow for the user to be aware of how much time has passed during a therapy session. For example, the timer 107 (e.g., by way of the control circuitry 102 and a mechanical vibration module) may cause the unit 10 to vibrate three times after 90 seconds have passed since the therapy session began, four times after two minutes have passed, and ten times after five minutes have passed. The device 10 may automatically shut off after a certain amount of time has passed (e.g., 2 minutes, 5 minutes, or somewhere in between). Alternatively, the device 10 may continue operating as long as the user wishes it to (though the device 10, if powered by battery, may shut off once the battery is depleted).

Referring to FIGS. 9-12, the user may engage the controller 75 to specify how much time passes between vibrations of the device 10B. The user may also engage the controller 75 to specify the duration of a therapy session. For example, since about two minutes of therapy may be the most advantageous to the user, the user may specify that the device 10B is to automatically shut off once two minutes have passed since the therapy session was initiated. Variations may be made to the temporal and temperature values discussed herein without departing from the scope of the present invention.

A temperature sensor 77 may be provided at the device 10B to monitor the user's body temperature to allow control circuitry of the device 10B to regulate the temperature of the thermally conductive faces 18 to achieve a desired body temperature, and/or to make sure user body temperature falls within a specified range. The temperature sensor 77 may comprise a thermometer at either conductive face 18, though the device 10B is not limited to any particular temperature sensor type, number and/or location. Alternatively or additionally, a temperature sensor may be provided separate from the device 10B, and the temperature sensor separate from the device 10B may be electronic communication with control circuitry of the device 10B. The device 10B may include a lithium battery 86 for powering various components thereof, though variations may be made to the means for powering the device 10B.

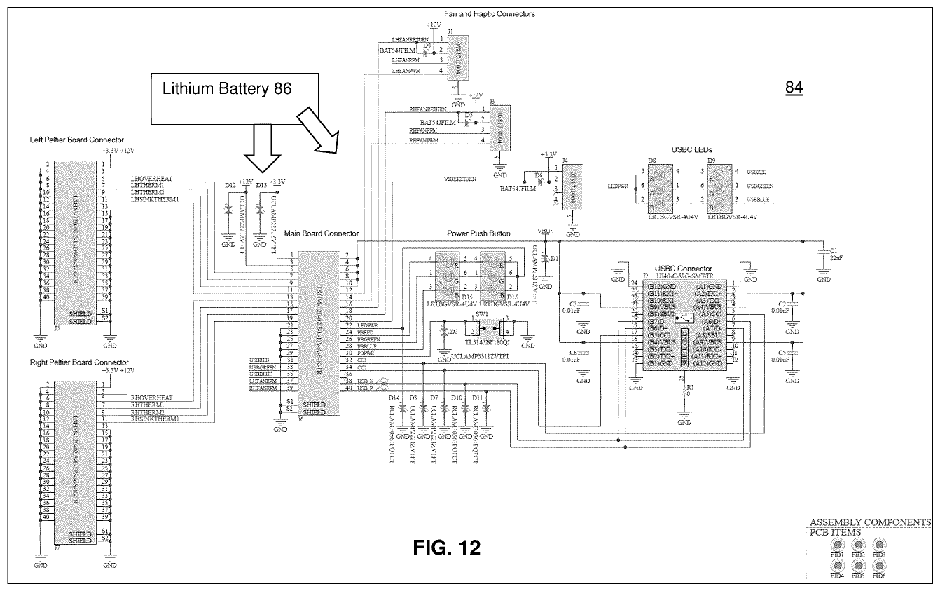

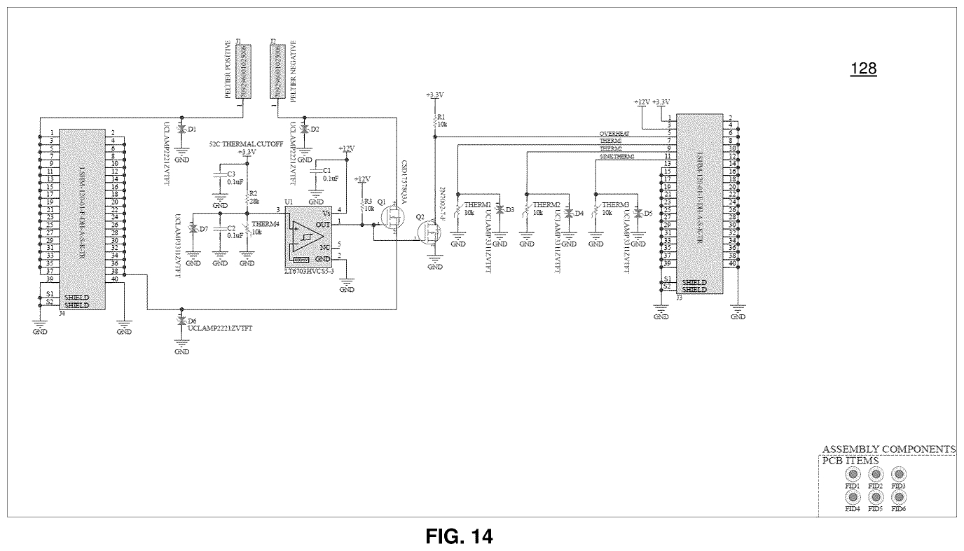

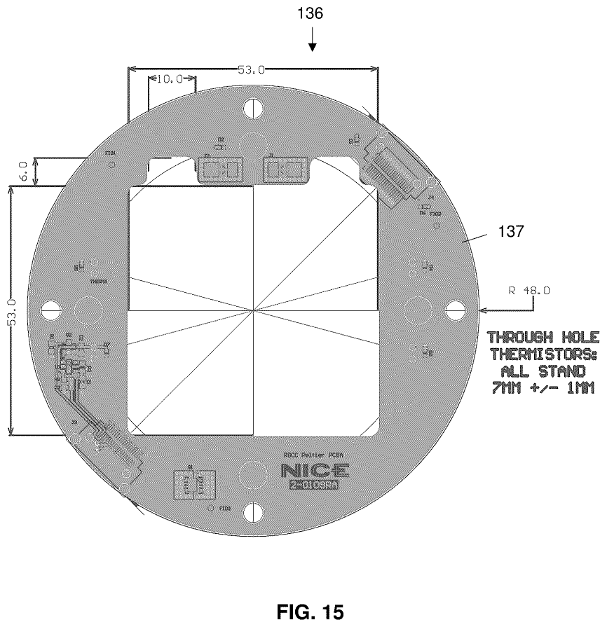

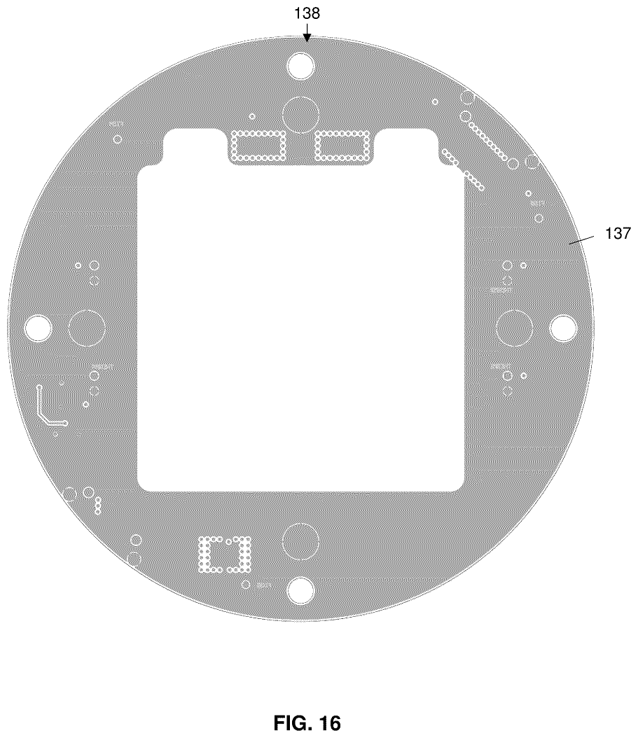

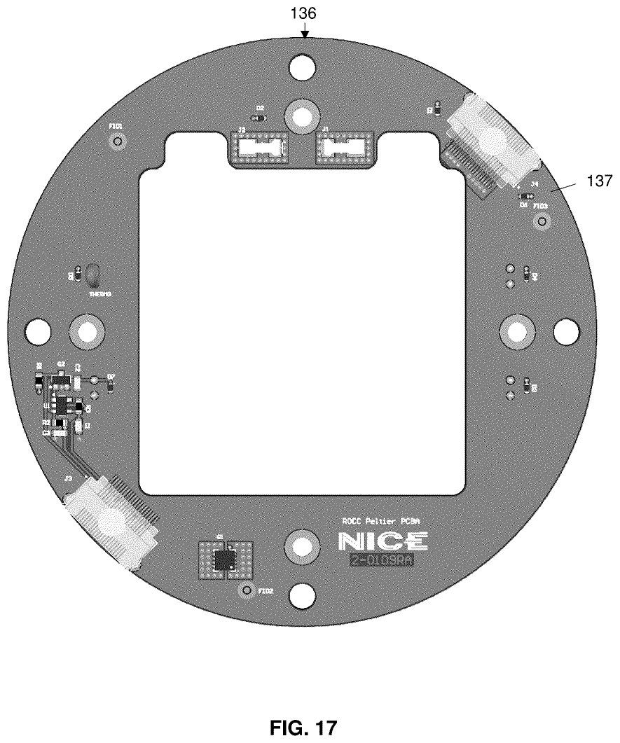

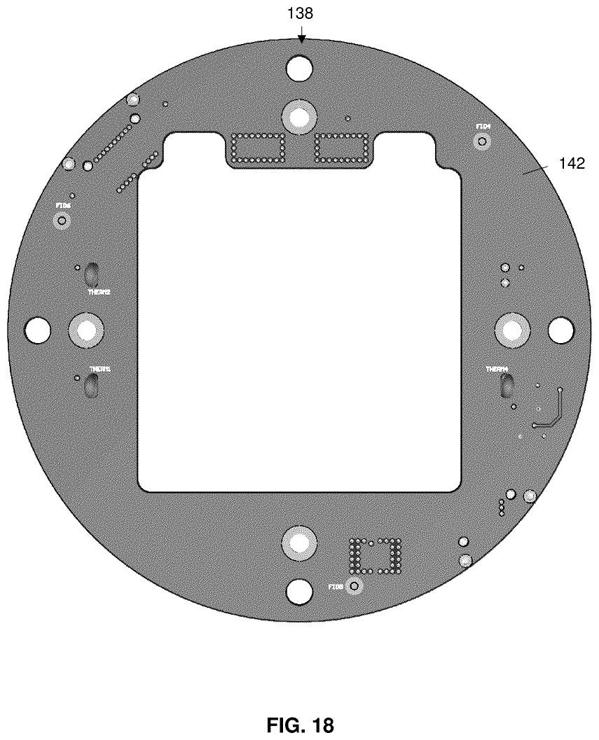

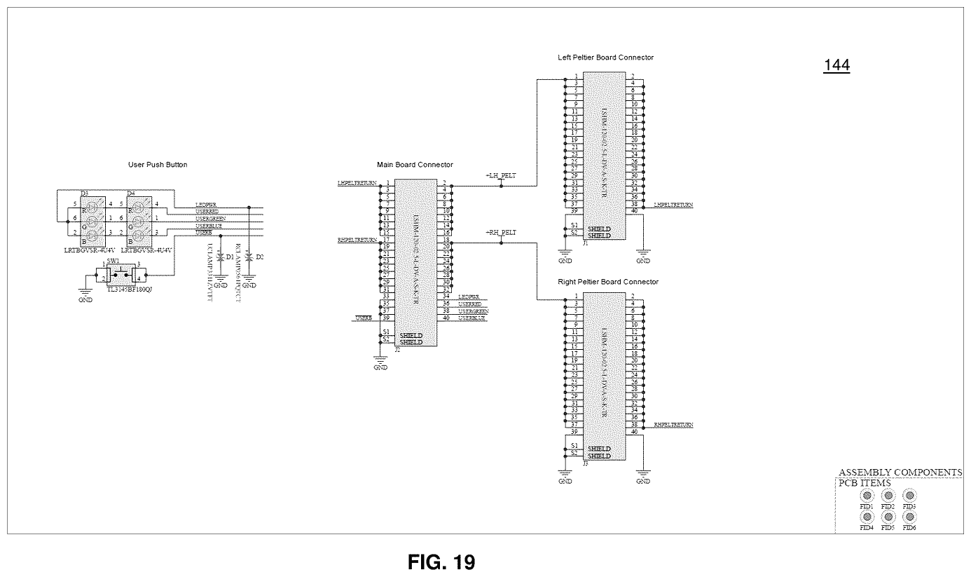

Referring to FIG. 12 and 14-19, exemplary circuitry schematics, PCBs, and PCBAs for an exemplary cooling therapy apparatus are shown. FIG. 12 specifically relates to exemplary circuitry 84 for powering on and off the device 10B of FIG. 9. FIGS. 14-18 specifically relate to exemplary circuitry 128 for one of two Peltier modules of the device 10B of FIG. 9. An exemplary device is not limited to two Peltier modules, or any particular number of temperature modification modules. The number and type of temperature modification modules may be varied. FIG. 19 specifically relates to control circuitry 144 of the device 10B of FIG. 9.

Referring now specifically to FIGS. 9 and 12, the hardware and circuitry 84 for powering on and off the device 10B may include a power button 80, one or more Peltier board connectors, fan and haptic connectors, main board connectors, USBC LEDs, USBC connectors, and the like. At least a portion of the circuitry 84 may be implemented by a printed circuit board assembly (“PCBA”) assembled from a printed circuit board (“PCB”). The power button 80, one or more Peltier board connectors, fan and haptic connectors, main board connectors, USBC LEDs, USBC connectors, and the like may each be in electronic communication with a power source. The power source may include a lithium battery 86. The battery 86 may be recharged by a charger (e.g., by way of a USBC port).

Referring to FIGS. 9 and 14-18, the Peltier module hardware and circuitry 128 of the device 10B may include Peltier board connectors, protection components, thermistor sensing paths, control integrated circuits, switching devices and the like configured to drive and protect components of the Peltier module. At least a portion of the circuitry 128 may be implemented by a PCBA 136 (e.g., having surface 137). The PCBA 136 may be assembled from a PCB 138 (e.g., having surfaces 137, 142). The Peltier module hardware and circuitry 128 components may each be in electronic communication with a power source.

Referring to FIGS. 9 and 19, the control hardware and circuitry 144 of the device 10B (e.g., for controlling a button board) may include a main board connector, left and right Peltier board connectors, a user push button interface, and the like configured to permit user control of the device 10B. At least a portion of the circuitry 144 may be implemented by a PCBA assembled from a PCB. The control hardware and circuitry 144 components may each be in electronic communication with a power source.

The PCBs may comprise one or more flat boards, copper traces, pads, holes, and the like. Electronic components mounted on the PCBs may include integrated circuits, resistors, capacitors, connectors, LEDs, and the like. Variations may be made to the hardware and circuitry illustrated and described herein without departing from the scope of the present invention. For example, the technique(s) for fabricating PCB(s) and/or assembling PCBA(s) of an exemplary device may be varied.

Referring back to FIG. 13, an exemplary method 90 for using a cooling therapy device 10 is shown. The device 10 may include an operable user interface 108 allowing a user 92 to regulate the temperature of one or more thermally conductive portions of the device 10. The user interface 108 may be located on an outer enclosure body portion of the device 10, and may comprise one or more buttons, dials, knobs, digital display screens (at and/or external to the device), some combination thereof, or the like. The user interface 108 may allow the user 92 to dictate the temperature of the thermally conductive portions over time. When the user interface 108 includes a digital display, said display may communicate information about the device 10 (e.g., battery state of charge, end face 18 temperature, and the like) to the user 92. One or more temperature modification modules 106 of the device 10 may be configured to control and adjust the temperature of the one or more thermally conductive portions. The device 10 may also include a temperature sensor 104 for monitoring a body temperature of the user 92 (e.g., at a glabrous region of the user, such as a palm). The temperature sensor 104 may be in electronic communication with the control circuitry 102, which may be configured to cause the temperature modification module(s) 106 (e.g., a Peltier module) to be adjusted when a measured body temperature of the user 92 by the sensor 104 falls outside of a predetermined temperature range.

The device 10 may also include a system control module 100. The system control module 100 may be configured to regulate the temperature of the one or more thermally conductive portions based on time passed during a cooling therapy session and/or temperature measurements of the temperature sensor 104 during the therapy session. The system control module 100 may include control circuitry 102, which may include and/or be linked to at least one processor 96. The control circuitry 102 may be in electronic communication with the user interface 108, and may be operable to actuate at least one power source 105 (e.g., one or more batteries) and the temperature modification module 106 (e.g., a Peltier module). The temperature modification module 106 may be configured to generate (e.g., for a finite time during a discrete cooling cycle initiated by the control circuitry 102) a cooler temperature on an exterior surface of a hemispherical portion of the device 10 than a temperature of ambient air. The device 10 may be adapted to generate a plurality of such discrete cooling cycles under the control of the user interface 108. The at least one processor 96 may be operatively coupled to a non-transitory memory storing system software 98. When executed by the processor 96, the system software 98 may cause the system control module 100 to regulate operation of various components (e.g., 106) of the device 10. The control circuitry 102 may additionally or alternatively include the non-transitory memory, related input and/or output interfaces, circuitry for said interfaces, a microcontroller, safety circuitry, logic circuitry, some combination thereof, or the like.

The control circuitry 102 may cause the one or more thermally conductive portions of the device 10 to reach a first temperature after a first amount of time, and to reach a second temperature after a second amount of time following the first amount of time. The second temperature may be warmer (or cooler) than the first temperature and cooler than the temperature of the ambient air. The second temperature may be cooler than 25° C. The temperature modification module(s) 106 may include a Peltier or other thermoelectric module secured with an outer enclosure of the device 10. The control circuitry 102 may be configured to cause a power supply to provide about 50-60 W (e.g., approximately 60 W) to the temperature modification module(s) 106 after a first amount of time (e.g., to achieve the first temperature), and to provide about 20-30 W (e.g., approximately 20-25 W) to the temperature modification module(s) after a second amount of time (e.g., to achieve the second temperature). The control circuitry 102 may automatically adjust the temperature of the one or more thermally conductive portions of the device 10 when a measured body temperature of the user 92 communicated to the control module 100 by the temperature sensor 104 falls above or below a threshold. One or more body temperature threshold values may be stored to the non-transitory, computer readable memory accessible by the software 98.

Referring to FIGS. 10-11 and 13, the control module 100 may dictate that, following device 10, 10B power-up (which may be caused by the user 92 engaging the user interface 108 and/or a power button 80), the amount of power supplied to the temperature modification module(s) 106 is approximately 60 W. Said amount of power may be supplied shortly after the device 10, 10B is powered on, and then the power supplied to the temperature modification module(s) 106 may be dropped to and maintained at approximately 20-25 W. The power supply values from table 82 (and/or other power supply values for regulating user 92's body temperature) may be stored to a non-transitory, computer readable memory accessible by the software 98. The user 92 (e.g., an athlete) may begin a cooling therapy session by grabbing the device 10, 10B with one or both palms of one's hands 74. Specifically, the user 92 may grasp thermally conductive, hemispherical end faces 18 of the device 10, 10B.

Thereafter, the control module 100 may cause the temperature of the end faces 18 to drop. Modulation 94 of the temperature of the end faces 18 may occur based on how much time has passed during the therapy session, and/or temperature measurements of the user 92 by the temperature sensor 104 of the device 10. Additionally, or alternatively, modulation 94 of the temperature of the end faces 18 may occur based on a detected surface area of cooling therapy. For example, when one or more apparatus sensors detect a contact surface area above a threshold for when the user 92 is grasping the device 10, the power supplied to the temperature modification module 106 may be automatically adjusted. The cooling therapy session may be terminated when a certain amount of time has passed, a minimum body temperature has been detected by the temperature sensor 104 (as a safety precaution), user input (e.g., pressing the power button 80) dictates the session is to be terminated, some combination thereof, or the like.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It will also be apparent to one of ordinary skill in the art that exemplary embodiments of the present invention are not necessarily intended to be limited to use with heat extraction for promoting muscle strength, endurance, performance, and the like. Exemplary embodiments of the present invention may also be useful for decreasing core body temperature to, e.g., address a fever, and/or for other purposes. It is the intention, therefore, to limit the invention only as indicated by the scope of the claims.

Certain operations described herein may be performed by one or more electronic devices. Each electronic device may comprise one or more processors, electronic storage devices, executable software instructions, and the like configured to perform the operations described herein. The electronic devices may be general purpose computers or specialized computing devices. The electronic devices may comprise personal computers, smartphones, tablets, databases, servers, processors, or the like, internal or external to the device, and when internal may be small or miniature size. The electronic connections and transmissions described herein may be accomplished by wired or wireless means.

What is claimed is:

1. A cooling therapy device, comprising:
a thermally conductive outer enclosure having at least one ambient air inlet and at least one air outlet;
an interior portion, comprising at least one temperature modification module secured within said outer enclosure;
wherein said at least one temperature modification module is configured to generate a cooler temperature at a first portion of said outer enclosure than a temperature of ambient air; and
wherein said device is configured to be hand-held, such that at least one of the palms of a user's hands may contact said first portion to cool the at least one palm.

2. The device of claim 1, further comprising a second temperature modification module configured to generate a cooler temperature at a second portion of said outer enclosure than said temperature of said ambient air, wherein said second portion is positioned opposite said first portion.

3. The device of claim 2, wherein said outer enclosure comprises a central body portion, a first end at one end of said central body portion, and a second end at another end of said central body portion, and wherein said first portion is at said first end and said second portion is at said second end.

4. The device of claim 3, wherein said first and second portions each comprise aluminum.

5. The device of claim 1, wherein said first portion is configured to cool a body temperature of a user for a limited period of time.

6. The device of claim 5, further comprising control circuitry configured to cause said first portion to reach a first temperature after a first amount of time, and to reach a second temperature after a second amount of time following said first amount of time, wherein said second temperature is warmer than said first temperature and cooler than said temperature of said ambient air.

7. The device of claim 3, wherein said first and second portions are each hemispherical in shape.

8. A system for providing cooling therapy, the system comprising:
a thermally conductive outer enclosure having at least one ambient air inlet and at least one air outlet;
an interior portion located within said outer enclosure, comprising:
a power supply secured within said outer enclosure;
at least one temperature modification module secured within said outer enclosure;
wherein said at least one temperature modification module is configured to generate a cooler temperature at a first portion of said outer enclosure than a temperature of ambient air; and
control circuitry configured to cause said first portion to reach a first temperature after a first amount of time.

9. The system of claim 8, wherein said control circuitry is configured to cause said first portion to reach a second temperature after a second amount of time following said first amount of time has passed, wherein said second temperature is warmer than said first temperature and cooler than said temperature of said ambient air.

10. The system of claim 8, wherein said at least one temperature modification module is configured to generate a cooler temperature at a second portion of said outer enclosure than said temperature of said ambient air.

11. The system of claim 10, wherein said first and second portions each comprise aluminum.

12. The system of claim 10, wherein said first and second portions are each hemispherical in shape.

13. The system of claim 9, wherein said second temperature is between about 50°F and about 60°F.

14. The system of claim 8, wherein said at least one temperature modification module is a thermoelectric module configured to produce a plurality of discrete cooling cycles, each cooling cycle occurring for a finite period of time, and said control circuitry is configured to cause said power supply to initially provide at least about 50 W to said thermoelectric module during one of said cooling cycles, and at a subsequent time in said one of said cooling cycles provide between about 20-30 W to said thermoelectric module.

15. A cooling therapy device, comprising:
an outer enclosure having a body portion and at least one hemispherical shaped distal end portion extending from said body portion, said at least one hemispherical shaped portion configured to be hand-held in the glabrous region of a user's palm;
an operable user interface on said outer enclosure body portion;
at least one ambient air inlet and at least one air outlet on said body portion;
at least one power source and at least one Peltier module secured within said outer enclosure;
control circuitry within said outer enclosure, in electronic communication with said user interface, operable to actuate said at least one power source and said at least one Peltier module;
wherein said at least one Peltier module is configured to generate a cooler temperature on an exterior surface of said at least one hemispherical portion than a temperature of ambient air; and
wherein said cooler temperature is generated for a finite time, during a discrete cooling cycle initiated by said control circuitry, and said device is adapted to generate a plurality of said discrete cooling cycles under control of said user interface.

16. The device of claim 15, further comprising a second hemispherical portion located opposite said first hemispherical portion, wherein said at least one Peltier module is configured to generate a cooler temperature at said second hemispherical portion of said outer enclosure than said temperature of said ambient air.

17. The device of claim 16, wherein said first and second hemispherical portions each comprise aluminum.

18. The device of claim 15, wherein said second temperature is between about 35°F and about 60°F.

19. The device of claim 15, wherein said second temperature is between about 50°F and about 60°F.

20. The device of claim 15, further comprising a temperature sensor in said device for monitoring a temperature of said glabrous region, and wherein said control circuitry is in electronic communication with said temperature sensor and is configured to cause said at least one Peltier module to be adjusted when said temperature sensor indicates the temperature of said glabrous region is outside a predetermined temperature range.
